(19) United States
(12) Patent Application Publication
(10) Pub. No.: US 2018/0030421 A1
(43) Pub. Date: Feb. 1, 2018

(54) Mixture of Purified SODs of Plant Origin

(71) Applicant: Bionov, Avignon (FR)

(72) Inventor: Dominique Lacan, Montpellier (FR)

(21) Appl. No.: 15/550,496

(22) PCT Filed: Feb. 12, 2016
(86) PCT No.: PCT/EP2016/052958

§ 371 Date: Aug. 11, 2017

(30) Foreign Application Priority Data
Feb. 12, 2015 (FR) 1551164

(51) Int. Cl. C12N 9/02

(52) U.S. Cl. CPC C12N 9/0089

(57) ABSTRACT

The present invention relates to an original and specific mixture of purified superoxide dismutases (SODs) of plant origin, characterised in that said mixture is essentially made up of three superoxide dismutases: a manganese superoxide dismutase, a copper and zinc superoxide dismutase and an iron superoxide dismutase provided in two isoforms, which can be obtained from an extract of the hybrid variety F1 of Cucumis melo MA 7950 or the cells thereof cultured in vitro or by transfer and expression of the genes of said SODs in prokaryotic or eukaryotic cells. The specific mixture according to the invention imparts to the compositions containing same a greater effectiveness in the treatment or prevention of diseases linked to inflammatory and/or oxidative stress, such as radiation-induced fibroses, cardiovascular diseases, obesity, atherosclerosis, labial herpes and myopathies, as well as in nutritional, pharmaceutical, veterinary or cosmetic uses.

The present invention relates to a mixture of superoxide dismutases (SODs) extracted from Cucumis melo, in particular the F1 hybrid melon variety called MA 7950, or from the cells thereof cultured in vitro or via transfer and expression of the genes of these SODs in prokaryote or eukaryote cells; to the preparation method thereof, to a cosmetic, nutritional, veterinary or pharmaceutical composition containing the same as active ingredient, and to said composition for use thereof as cosmetic care product, food supplement, beverage or medicinal product.

Oxygen, that is essential for body function, nevertheless generates toxic reactive oxygenated forms which have a negative effect on the body. These reactive oxygenated forms are mostly free radicals such as the superoxide radical (O₂.—), hydroxyl radical (HO.), nitrogen monoxide (.NO) or peroxide radicals of lipid origin (L-OO.). The superoxide ion is the most abundant among reactive oxygen species and it is directly formed from oxygen; all the other reactive oxygen species originate therefrom, in particular the hydroxyl radical (HO.) which is most aggressive.

These free radicals are atoms or molecules, the electronic configuration of which is characterized by the presence of a non-paired electron. This particularity imparts instability thereto, and to stabilise themselves they may rapidly oxidize other biological molecules such as nucleic acids (DNA), enzymatic proteins or membrane lipids, and in particular polyunsaturated fatty acids (PUFAs).

Our body constantly acts against the formation of reactive oxygen species which destroy cells. All cells have constituent lines of defence for cell detoxification.

The first line of defence is ensured by an enzyme called superoxide dismutase (SOD) which is a metalloenzyme (Mac Cord and Fridovich, J. Biol. Chem. 244: 6049-55, 1969). In the plant kingdom, there are three forms of SODs (Michalski, J. Chromatogr. B 684: 59-75, 1996), each one characterized by the presence of a metal ion positioned at its active site:
one form of SOD contains copper and zinc (Cu/Zn-SOD) and is chiefly located in the cytoplasm and chloroplast;
another form contains manganese (Mn-SOD) and is located in the mitochondria and peroxisome;
and finally, in some plant species a SOD contains iron (Fe-SOD) and is located in the chloroplast.

SODs play a key role in the fight against free radicals since they allow elimination of the superoxide ion. Superoxide dismutases are enzymes capable of inducing dismutation of superoxide ions as per the following reaction:

2O₂.⁻+2H₂O→O₂+H₂O₂+2OH⁻ (equation 1).

The action of SODs is completed by a second line of defence which eliminates H₂O₂: catalase and/or selenium-dependent glutathione peroxidase (SeGPx), which ensure destruction of hydrogen peroxide H₂O₂.

Under normal biological conditions, the human body constantly produces free radicals in small amounts which are immediately neutralised by existing defence systems. In some cases, if the production of free radicals is increased (tobacco, stress, pollution, sun rays, unbalanced diet . . . ), and/or if there is a deficiency of antioxidant substances, this leads to oxidant/antioxidant imbalance inducing major cell changes in the macromolecules (oxidation of DNA, proteins, sugars, carbohydrates) but also in cell organelles in particular the mitochondria, the main conveyors of energy for the cells which, if deteriorated, become a major source of free radicals. This imbalance has an increasingly close correlation with numerous pathologies and numerous imbalances. SOD, by neutralising the superoxide anion—the cause of all reactive oxygen species and hence of oxidative stress—plays a key role in regulating oxidative stress, and the administering of exogenous SOD is contemplated in the treatment of pathologies related to oxidative stress.

Up until now, the only purified SODs available have been derived from the animal kingdom, extracted in particular from bovine erythrocytes (Markovitz, J. Biol. Chem., 234, p. 40, 1959), from Escherichia Coli (Keele and Fridovitch, J. Biol., 245, p. 6176, 1970) and from marine bacterial strains (patents FR 2 225 443 and FR 2 240 277).

SOD, chiefly the bovine Cu—Zn-SOD form, has been the subject of pharmaceutical development leading to a medicinal product with the name Orgotein®. It has been used in pathologies induced by free radicals, in particular in cases of chronic inflammation such as in the treatment of Crohn's disease (Emerit et al., 1991, Free Rad. Res. Comms, 12-13, 563-569) or for radiotherapy-induced fibrosis (Delanian et al. 1994, Radiotherapy and Oncology, 32, 12-20).

Nevertheless, for reasons mainly related to infectious substances which may be contained in such materials, these animal SODs at least in France were soon prohibited in the 1990s. On this account, the search for a SOD source derived from the plant kingdom has been the focus of increasing interest in the pharmaceutical field.

Alternative compositions comprising SOD of plant origin, of melon in particular, have therefore been proposed. The benefits drawn from the use of melon-based food supplements are well known as illustrated in the article by Milind et al (International Research Journal of Pharmacy, 2011, 8, 52-57), the article by Voudoukis and Lacan et al (Journal of Ethnopharmacology 2004, 94, 67-75), or the article by Gene et al (Journal of Agricultural and Food Chemistry, 2008, 56, 3694-3698).

In addition, melons generally have a particularly high SOD content compared with other fruit. The SOD extraction yield from melons is therefore high and economically profitable. It is also to be noted that extracts from other fruit having a sufficiently high content for observation of effects due to the presence of SOD, are not yet commercially available.

Formulations of SODs of plant origin, and in particular from melon, coated with a wheat gliadin matrix have been described e.g. in patent application FR 2 729 296, or the product GLISODIN®, for the purpose of protecting SOD-containing compositions intended for administration via oral route against gastric juices.

Also, patent FR 2 716 884 describes a protein extract of Cucumis melo, preferably the variety 95LS444, having higher superoxide dismutase enzymatic action at 30 units/mg of soluble proteins, the method for preparation thereof and use thereof in pharmaceutical or cosmetic compositions for external topical use. However, said protein extract, although rich in superoxide dismutase, cannot be used in numerous pharmaceutical applications in particular because it is not sufficiently purified, nor sufficiently active. It is indicated in Example 1 that the protein extract of Cucumis Melo of the 95LS444 variety has a superoxide dismutase enzymatic activity of 126 U/mg proteins. Said extract, derived from the Clipper melon (a descendant of the Cucumis Melo 95LS444 variety) is also described with the same overall SOD content in Lacan et al (Planta 1998, 204, 377-382). It will be noted that this article describes a single isoform of Fe-SOD.

There is therefore a need to obtain a mixture of SODs of plant origin having improved pharmaceutical activity to combat diseases related to oxidative stress and the consequences thereof, in particular cardiovascular diseases and obesity and the consequences thereof.

Surprisingly, the Applicant has found a mixture of SODs of plant origin having higher antioxidant properties than SOD mixtures in the prior art. In particular, the SOD mixture of the invention is more active than a SOD mixture derived from a protein extract of the 95LS444 variety of Cucumis Melo or one of the descendants thereof such as the Clipper melon.

Without being limited to this interpretation however, it would seem that the presence in said mixture of a particular isoform of iron superoxide dismutase, which has never been described up until now, is able to impart its greater pharmaceutical properties to the mixture of the invention.

The present invention therefore firstly concerns a mixture of superoxide dismutases of plant origin, essentially consisting of 3 superoxide dismutases.

The mixture of SODs of the invention has surprising properties and beneficial effects on the cardiovascular system, in particular on cardiac hypertrophy. The mixture of SODs of the invention surprisingly modulates the expression of some genes associated with heart pathologies and obesity, such as the NPPA or RXFP1 gene.

The present invention also concerns an isoform of iron SOD (Fe-SOD) of plant origin having antioxidant activity of particular interest.

The present invention also concerns a method to prepare said mixture of superoxide dismutases of plant origin, essentially consisting of 3 superoxide dismutases.

The present invention also concerns a method to prepare said isoform of iron SOD (Fe-SOD) of plant origin having antioxidant activity of particular interest.

The present invention also concerns a cosmetic, nutritional, veterinary or pharmaceutical composition which, as active ingredient, contains a mixture of purified superoxide dismutases of the invention or the isoform of iron SOD (Fe-SOD) of plant origin according to the invention, and at least one pharmaceutically or cosmetically acceptable food-grade excipient.

The present invention also concerns a composition of the invention for use thereof as medicinal product.

The present invention therefore firstly concerns an original mixture of superoxide dismutases of plant origin, essentially consisting of 3 superoxide dismutases: a manganese superoxide dismutase, a copper and zinc superoxide dismutase and an iron superoxide dismutase in at least two isoforms, the first isoform of iron superoxide dismutase having a molecular weight of between 28 000 and 36 000 Da, the second isoform of iron superoxide dismutase having a molecular weight of between 75 000 and 85 000 Da, said mixture able to be obtained from an extract of the F1 hybrid variety of Cucumis Melo MA 7950 or the cells thereof cultured in vitro or via transfer and expression of the genes of these SODs in prokaryote or eukaryote cells, and said mixture preferably having a total SOD activity equal to or higher than 130 U/mg of the mixture. Preferably, said mixture is able to be obtained from an extract of the F1 hybrid variety of Cucumis Melo MA 7950 or the cells thereof cultured in vitro, more preferably said mixture is able be obtained from an extract of the F1 hybrid variety of Cucumis Melo MA 7950. It is noted on passing that the Cucumis Melo descending from the MA 7950 cell line, or from one of the hybrid varieties derived from MA 7950, have a particularly high SOD content, both compared with other fruit and compared with other melon varieties. Therefore, the SOD extraction yield from these specific Cucumis Melo is particularly high and economically profitable.

By “essentially consisting of” in the meaning of the present invention is meant that the mixture comprises between 70 and 99.9%, advantageously 80 to 99.9% by weight of superoxide dismutases. The other constituents of the mixture have a minimum impact on its enzymatic activity. Preferably, the other constituents do not interfere with superoxide dismutase enzymatic activity.

Preferably, the mixture of the invention is extracted from the F1 hybrid variety of Cucumis Melo MA 7950, or the cells thereof cultured in vitro or via transfer and expression of the genes of these SODs in prokaryote or eukaryote cells. More preferably, the mixture of the invention is extracted from the F1 hybrid variety of Cucumis Melo MA 7950, or the cells thereof cultured in vitro.

It will be noted that persons skilled in the art with their general knowledge are able to obtain the peptide sequence of the SODs of the mixture of the invention, and using well known techniques are then able to generate the corresponding messenger RNA (mRNA) and complementary DNA (cDNA). The latter can be transferred using well known techniques into prokaryote or eukaryote cells which are subsequently used to produce the SOD mixture of the invention.

The “Cucumis Melo MA 7950 cells cultured in vitro”, in the meaning of the present invention, comprise both cells that are directly derived from the plant and cultured in vitro using techniques well known to skilled persons, particularly stem cells, and cells derived from these first cells.

The F1 hybrid variety of Cucumis melo MA 7950, the seeds of which were deposited with the NCIMB collection (National Collection of Industrial and Marine Bacteria—ABERDEEN AB21 9YA (Scotland—GB) Ferguson Building Craibstone Estate Bucksburn) on 8 Jul. 2013 under number NCIMB 42154—conforming to the Treaty of Budapest—has unique characteristics regarding its appearance, its stress resistance and SOD composition. This SOD mixture has greater antioxidant properties than other mixtures of SODs derived from other plant sources, and derived from other melon varieties in particular.

By “extract” in the meaning of the present invention is meant a protein extract, preferably a soluble protein extract.

By “SOD” in the meaning of the present invention is meant an enzyme of superoxide dismutase type. It is to be noted that the superoxide dismutases of the invention are natural i.e. they are not chemically modified. In particular, the present invention concerns SODs in their entirety and not fragments thereof. SODs are classified into three categories according to the metal contained at their active site: manganese superoxide dismutases (Mn-SOD), copper and zinc superoxide dismutases (Cu/Zn-SOD) and iron superoxide dismutases (Fe-SOD).

In the mixture of the present invention, there are at least two isoforms of Fe-SOD. The isoforms of a protein are the different forms assumed when derived from different genes, or from the same gene by alternate splicing. In the remainder hereof, these two isoforms of Fe-SOD will be called “first isoform” and “second isoform” as a function of their molecular weight. Therefore the “first isoform” of the invention has a lower molecular weight than the “second isoform”.

It is to be noted that the other SODs (Mn-SOD and/or Cu/Zn-SOD) may also be contained in the mixture in several isoforms. Therefore, according to the present invention, the term Mn-SOD (respectively Cu/Zn-SOD) covers all the isoforms of Mn-SOD (respectively Cu/Zn-SOD) contained in the mixture.

SODs catalyse the dismutation of superoxide ions as per the reaction:

2O₂.⁻+2H₂O→O₂+H₂O₂+2OH⁻ (equation 1).

The mixture of the invention is characterized by its total SOD activity. By “total SOD activity” in the meaning of the present invention is meant the quantification of the dismutation reaction described in equation 1 by the mixture of the invention. This total SOD activity is measured using techniques well known to skilled persons and expressed in U (enzymatic unit or unit) per mg of proteins. Preferably, it is measured using a method based on the reduction of tetrazolium salt. In particular, the method of Beauchamp and Fridovich can be cited (Anal. Biochem. 44 :276-82 (1971)) based on inhibition by SOD of the reduction of Nitroblue Tetrazolium (N 55 14 Sigma-Aldrich, France), and modified by Oberley and Spitz in 1985 (Boca Raton CRC Press; In R.A. Grenwald 1985, ed: Handbook of Methods for oxygen radical research, p 217-230). It is also possible to use the SOD assay kit (19160, Sigma-Aldrich, France), also based on the reducing of tetrazolium salt, this being a variant of the preceding method but easier to implement.

The SOD mixture of the invention preferably a total SOD activity higher than 130 U/mg of said mixture, more preferably higher than 300 U/mg of said mixture, most preferably higher than 500 U/mg of said mixture.

In addition, it is also possible to determine the SOD activity of each SOD (Mn-SOD, Cu/Zn-SOD and Fe-SOD). This “relative” SOD activity quantifies the contribution of each SOD (or one of the isoforms thereof) to the total SOD activity of the mixture. It is therefore expressed as a percentage of the total SOD activity of the mixture. The SOD activity of each SOD (or one of the isoforms thereof) is measured using techniques well known to persons skilled in the art. The different SOD forms can be identified using different inhibitors: KCN inhibits the activity of Cu/Zn SOD; H₂O₂ inhibits the activity of Fe SOD and Cu/Zn SOD, while Mn SOD is insensitive to H₂O₂ and KCN (Paul and Van Alstyne, J Exp. Mar. Biol. Ecol. 160: 191-203 1992).

Therefore, first the total SOD activity of the mixture is measured.

The method to determine the different forms of SOD, well known to skilled persons, comprises separation by electrophoresis on acrylamide gel under native conditions, followed by detection of SOD activity on the gel.

Samples of the SOD mixture of the invention are deposited in the different wells of electrophoresis gel. Migration under non-denaturing conditions takes place. At the gel detection step the gel is cut into 3 portions. The first detects total SOD activity, the second is preincubated with 2 mM KCN and determined for SOD activity under the same conditions, finally the last is preincubated with 5 mM H₂O₂ and SOD activity is determined under the same conditions. The intensities of the different bands are measured by integration of the area under curve, and their ratio indicates the “relative” SOD activities of the different SODs (or the isoforms thereof) in the mixture of the invention.

An example of the determination of “relative” SOD activity of the different SODS in the mixture is illustrated in the examples.

Therefore, the mixture of superoxide dismutases of the invention is advantageously such that the accumulated SOD activity of the two isoforms of iron superoxide dismutase is between 20% and 26%, advantageously between 22% and 26% of the total SOD activity of the mixture.

According to one preferred variant of the invention, the mixture of purified superoxide dismutases is such that the accumulated SOD activity of the two isoforms of iron superoxide dismutase is between 20% and 26%, advantageously it is 25% of the total SOD activity of the mixture, the activity of the copper and zinc superoxide dismutase is between 60% and 70%, advantageously it is 65% of the total SOD activity of the mixture, and the activity of the manganese superoxide dismutase is between 9 and 15%, advantageously between 7 and 12%, more advantageously it is 10% of the total SOD activity of the mixture.

SODs, like all proteins, can be separated as a function of two biochemical characteristics: their molecular weight and isoelectric point.

The separation of the different forms of SOD can be performed by acrylamide gel electrophoresis under native conditions, followed by detection of SOD activity on the gel.

Visualisation of the different SODs and measurement of SOD activity are conducted using the method of Beauchamp and Fridovich (Anal. Biochem. 44 :276-82 (1971).

The molecular weights of the different SOD forms can be determined using molecular weight markers (proteins of known molecular weight) to calibrate the electrophoresis gel. The different SOD forms are identified as described above and the position of the bands obtained is compared with those of the molecular weight markers to determine the molecular weight of each SOD isoform.

Therefore, preferably, the mixture of the invention comprises an iron superoxide dismutase contained in at least two isoforms, the first having a molecular weight of between 28 000 and 36 000 Da, and the second having a molecular weight of between 75 000 and 85 000 Da.

Advantageously, the first isoform of iron superoxide dismutase in the SOD mixture of the invention has a molecular weight of about 32 200 Da.

Advantageously, the second isoform of iron superoxide dismutase in the SOD mixture of the invention has a molecular weight of about 79 800 Da.

In one preferred embodiment, the manganese superoxide dismutase has a molecular weight of between 70 000 and 90 000 Da, and the copper and zinc superoxide dismutase has a molecular weight of between 27 000 and 35 000 Da.

According to one preferred variant of the invention, the Cu/Zn-SOD has a molecular weight of between 27 000 and 35000 Da, advantageously it is 31 800 Da; Mn-SOD has a molecular weight of between 70 000 and 90 000 Da, advantageously it is 80 600 Da.

For the Fe-SODs, the first isoform has a molecular weight of between 28 000 and 36 000 Da, advantageously it is 32 200 Da, the second Fe-SOD isoform has a molecular weight of between 75 000 and 85 000 Da, advantageously it is 79 800 Da.

The separation of SODs as a function of their charge is performed by isoelectric focusing (IEF). SODs migrate in an electric field and become immobilised when they no longer have any net charge due to the pH environment. The isoelectric points (or pHi) of the different SOD forms can be determined on IEF gels by comparison with known isoelectric point markers (IEF markers 3.6-9.3, Reference 56733, Sigma-Aldrich, France).

By isoelectric point, or pHi, in the meaning of the present invention is meant the pH at which the net charge of this molecule is zero or, in other words, the pH at which the molecule is electrically neutral.

Preferably, Cu/Zn-SOD has a pHi of 4.3.

Advantageously, the two isoforms (the first and second) of Fe-SOD have a pHi of 4.4 and 4.7 respectively.

According to one preferred variant of the invention, Cu/Zn-SOD has a pHi of 4.3, the Fe-SODs have a pHi of 4.4 and 4.7 and the different isoforms of Mn SOD have pHi values of 4.1; 4.4; 5.3; 5.5; 5.7; 5.85; 6.1.

Additionally, the present invention concerns a method to prepare the mixture of superoxide dismutases of the invention. Said method comprises the successive steps of:
grinding or pressing in an aqueous medium, preferably at a pH of 5 to 9, the F1 hybrid variety of Cucumis Melo MA 7950 or the cells thereof cultured in vitro or via transfer and expression of the genes of these SODs in prokaryote or eukaryote cells.
recovering the supernatant, and
purifying by chromatography, in particular by IMAC chromatography (Immobilized Metal Ion Affinity chromatography).

Therefore, the mixture of superoxide dismutases of the invention, preferably purified, is able to be obtained with the method comprising the successive steps of:
grinding or pressing in an aqueous medium, preferably at a pH of 5 to 9, the F1 hybrid variety of Cucumis Melo MA 7950 or the cells thereof cultured in vitro or via transfer and expression of the genes of these SODs in prokaryote or eukaryote cells,
recovering the supernatant, and
purifying by chromatography, in particular IMAC chromatography

(“Immobilized metal ion affinity chromatography”)

Immobilized metal ion affinity chromatography (IMAC) is based on the affinity of some amino acids, in particular histidine, tryptophan and cysteine, for metals. This chromatographic technique therefore operates by enabling proteins having an affinity for metal ions to be retained on a column containing immobilised metal ions such as cobalt, nickel, copper. The eluents used generally have a pH gradient, or concentration of competitive molecule, for binding to the ions of the column, such as imidazole.

Preferably, IMAC chromatography is applied using a column comprising copper ions as immobilised metal ions. Therefore, the eluting rate of the proteins is dependent on their charge and on their affinity for the metal ion, copper in particular, and is hence correlated with the presence of amino acids such as tryptophan, histidine, cysteine in the protein sequence. Advantageously, as eluent, a gradient of aqueous solutions of NH₄Cl and CuSO4 is used.

Alternatively, the method of the invention comprises the successive steps of:
grinding or pressing in an aqueous medium, preferably at a pH of 5 to 9, the F1 hybrid variety of Cucumis Melo MA 7950 or the cells thereof cultured in vitro or via transfer and expression of the genes of these SODs in prokaryote or eukaryote cells,
recovering the supernatant, and
successive membrane purifications in particular by passing the supernatant through membranes of varying porosity.

Persons skilled in the art are able to adapt the porosity gradient of the membranes for the method as a function of the ripeness and dry matter content of the melon, in particular by seeking to avoid clogging of the membranes.

As indicated above, it would seem that the presence of the second isoform of iron superoxide dismutase, which has never been described to date, is able to impart its higher biological properties to the mixture of the invention.

This second isoform of iron superoxide dismutase, able to be obtained from an extract of the F1 hybrid variety of Cucumis Melo MA 7950, has a molecular weight of between 75 000 and 85 000 Da.

Therefore, more generally, the present invention concerns a (synthetic or natural) superoxide dismutase SODᵢ, the polypeptide sequence of which is homologous with the sequence of the second isoform, and in particular has a sequence identity equal to or higher than 80%, preferably 85%, 90%, 95% or 98%, with the sequence of the second isoform.

A homologous polypeptide sequence of the second isoform includes any polypeptide sequence which differs from the sequence of the second isoform by mutation, insertion, deletion or substitution of one or more amino acids, provided it has the biological activity of the second isoform.

By “percent identity” between two sequences of amino acids in the meaning of the present invention, it is meant to designate a percentage of identical amino acid residues between the two sequences to be compared, obtained after the best alignment (optimal alignment), this percentage being purely statistical and the differences between the two sequences being randomly distributed over their entire length. Sequence comparison between two sequences of amino acids is conventionally performed by comparing these sequences after optimal alignment thereof, said comparison possibly being conducted per segment or per “comparison window”. The optimal aligning of sequences for comparison can be obtained, other than manually, by using the local homology algorithm of Smith and Waterman (1981) [Ad. App. Math. 2:482], using the local homology algorithm of Neddleman and Wunsch (1970) [J. Mol. Biol. 48:443], using the similarity search method of Pearson and Lipman (1988) [Proc. Natl. Acad. Sci. USA 85:2444], using computer software applying these algorithms (GAP, BESTFIT, FASTA and TFASTA in Wisconsin Genetics Software Package, Genetics Computer Group, 575 Science Dr., Madison, Wis.) or using BLAST N or BLAST P comparison software.

The percent identity between two sequences of amino acids is determined by comparing these two optimally aligned sequences, wherein the sequence of amino acids to be compared may comprise additions or deletions relative to the reference sequence for optimal alignment between these two sequences. Percent identity is calculated by determining the number of identical positions at which the amino acid residue is identical between the two sequences, by dividing this number of identical positions by the total number of positions in the comparison window, and multiplying the result obtained by 100 to obtain the percent identity between these two sequences.

For example, the BLAST programme “BLAST 2 sequences” can be used (Tatusova et al., “Blast 2 sequences—a new tool for comparing protein and nucleotide sequences”, FEMS Microbiol., 1999 Lett. 174:247-250) available on the website http://www.ncbi.nlm nih.gov/gorf/b12.html, the parameters used being those given by default (in particular for the “open gap penalty”: 5, and “extension gap penalty”: 2 parameters; the chosen matrix for example being the “BLOSUM 62” matrix proposed in the programme), the percent identity between the two sequences to be compared being calculated directly by the programme.

By “amino acid” in the meaning of the present invention is meant all residues of natural α-amino acids (e g Alanine (Ala), Arginine (Arg), Asparagine (Asn), Aspartic acid (Asp), Cysteine (Cys), Glutamine (Gln), Glutamic acid (Glu), Glycine (Gly), Histidine (His), Isoleucine (Ile), Leucine (Leu), Lysine (Lys), Methionine (Met), Phenylalanine (Phe), Proline (Pro), Serine (Ser), Threonine (Thr), Tryptophan (Trp), Tyrosine (Tyr) and Valine (Val)) in D or L form, and non-natural amino acids (e.g. β-alanine, allylglycine, tert-leucine, 3-amino-adipic acid, 2-aminobenzoic acid, 3-aminobenzoic acid, 4-aminobenzoic acid, 2-aminobutanoic acid, 4-amino-1-carboxymethyl piperidine, 1-amino-1-cyclobutanecarboxylic acid, 4-aminocyclohexaneacetic acid, 1-amino-1-cyclohexanecarboxyilic acid, (1R,2R)-2-aminocyclohexanecarboxylic acid, (1R,2S)-2-aminocyclohexanecarboxylic acid, (1S ,2R)-2-aminocyclohexanecarboxylic acid, (1S,2S)-2-aminocyclohexanecarboxylicacid, 3-aminocyclohexanecarboxylic acid, 4-aminocyclohexanecarboxylic acid, (1R,2R)-2-aminocyclopentanecarboxylic acid, (1R,2S)-2-aminocyclopentanecarboxyilic acid, 1-amino-1-cyclopentanecarboxylic acid, 1-amino1-cyclopropanecarboxylic acid, 4-(2-aminoethoxy)-benzoic acid, 3-aminomethylbenzoic acid, 4-aminomethylbenzoic acid, 2-aminobutanoic acid, 4-aminobutanoic acid, 6-aminohexanoic acid, 1-aminoindane-1-carboxylic acid, 4-aminomethyl-phenylacetic acid, 4-aminophenylacetic acid, 3-amino-2-naphtoic acid, 4-aminophenylbutanoic acid, 4-amino-5-(3-indolyl)-pentanoic acid, (4R,5S)-4--amino-5-methylheptanoic acid, (R)-4-amino-5-methylhexanoic acid, (R)-4-amino-6-methylthiohexanoic acid, (S)-4-amino-pentanoic acid, (R)-4-amino-5-phenylpentanoic acid, 4-aminophenylpropionic acid, (R)-4-aminopimeric acid, (4R,5R)-4-amino-5-hyroxyhexanoic acid, (R)-4-amino-5-hydroxypentanoic acid, (R)-4-amino-5-(p-hydroxyphenyl)-pentanoic acid, 8-aminooctanoic acid, (2S,4R)-4-amino-pyrrolidine-2-carboxylic acid, (2S,4S)-4-amino-pyrrolidine-2-carboxylic acid, azetidine-2-carboxylic acid, (2S,4R)-4-benzyl-pyrrolidine-2-carboxylic acid, (S)-4,8-diaminooctanoic acid, tert-butylglycine acid, γ-carboxyglutamate, β-cyclohexylalanine, citrulline, 2,3-diamino propionic acid, hippuric acid, homocyclohexylalanine, moleucine, homophenylalanine, 4-hydroxyproline, indoline-2-carboxylic acid, isonipecotic acid, α-methyl-alanine, nipecotic acid, norleucine, norvaline, octahydroindole-2-carboxylic acid, ornithine, penicillamine, phenylglycine, 4-phenyl-pyrrolidine-2-carboxylic acid, pipecolic acid, propargylglycine, 3-pyridinylalanine, 4-pyridinylalanine, 1-pyrrolidine-3-carboxylic acid, sarcosine, statins, tetrahydroisoquinoline-1-carboxylic acid, 1,2,3,4-tetrahydroisoquinoline-3-carboxylic acid, tranexamic acid). The term also includes natural and non-natural amino acids carrying a conventional amino-protective group (e.g. an acetyl, tert-butyloxycarbonyl, benzyloxycarbonyl or 9-fluorenylmethylcarbonyl group), and natural and non-natural amino acids protected at the carboxylic end (advantageously by a C1-C18 alkyl group, an ester, a phenyl or benzyl amide or an amide, which respectively gives carboxylic ends of following formulas: —CO(C1-C18 alkyl), —COO(C1-C18 alkyl), —CONHphenyl CONHbenzyl or CONH2.

By amino acid sequence having at least 80%, preferably 85%, 90%, 95% and 98% identity with a reference amino acid sequence, preference is given to those having some modifications relative to the reference sequence, in particular a deletion, addition or substitution of at least one amino acid, a truncation or extension. For a substitution of one or more consecutive or non-consecutive amino acids, preference is given to substitutions in which the substituted amino acids are replaced by “equivalent” amino acids. The expression “equivalent amino acids” herein designates any amino acid able to be substituted for one of the amino acids of the base structure without however essentially modifying the biological activities of the corresponding antibodies and such as defined in the remainder hereof, in particular in the examples.

These equivalent amino acids can be determined for example by referring to their structural homology with the amino acids that they substitute.

This search for similarity in a polypeptide sequence takes into account conservative substitutions which are substitutions of amino acids in the same class, such as substitutions of amino acids with non-charged side chains (e.g. asparagine, glutamine, serine, threonine and tyrosine), of amino acids with basic side chains (e.g. lysine, arginine and histidine), of amino acids with acidic side chains (e.g. aspartic acid and glutamic acid); of amino acids with polar side chains (e.g. glycine, alanine, valine, leucine, isoleucine, proline, phenylalanine, methionine, tryptophan and cysteine).

Preferably, the “variant”, “homologous” or “derived” superoxide dismutases have the same length as the reference sequences. Therefore, advantageously, the sequence of the superoxide dismutase of the invention SOD, consists of the sequence of the second isoform of iron superoxide dismutase able to be obtained from an extract of the F1 hybrid variety of Cucumis Melo MA 7950 and having a molecular weight of between 75 000 and 85 000 Da.

In particularly advantageous manner, the present invention concerns the second isoform of the iron superoxide dismutase able to be obtained from an extract of the F1 hybrid variety of Cucumis Melo MA 7950 and having a molecular weight of between 75 000 and 85 000 Da. It can be obtained with a method comprising the successive steps of:
grinding or pressing in an aqueous medium, at a pH of 5 to 9, the F1 hybrid variety of Cucumis Melo MA 7950 or the cells thereof cultured in vitro or via transfer and expression of the genes of these SODs in prokaryote or eukaryote cells,
recovering the supernatant, and
purification by chromatography in particular IMAC chromatography.

The present invention also concerns any mixture comprising said superoxide dismutase SODᵢ.

The present invention also concerns a cosmetic, nutritional, veterinary or pharmaceutical composition containing, as active ingredient, the superoxide dismutase SODᵢ or the mixture of superoxide dismutases of the invention, and at least one food-grade or pharmaceutically or cosmetically acceptable excipient.

In the meaning of the present invention, the term “pharmaceutical composition” covers both a composition for pharmaceutical or veterinary use i.e. it is intended to be used to treat an animal, including man.

By “animal” in the meaning of the present invention is meant for example a mammal, fish, shellfish, reptile, bird. In particular, a mammal comprises man.

In the meaning of the present invention, the term “nutritional composition” particularly encompasses nutraceutical compositions (particularly food supplements for example in solid or liquid form), health-food compositions and beverages, particularly of dietary or nutritional nature such as beverages with antioxidant properties. Preferably, a nutritional composition comprises nutraceutical compositions and health beverages such as beverages with antioxidant properties.

In the present description, by food-grade or pharmaceutically or cosmetically acceptable excipient is meant a compound or combination of compounds included in a nutritional, pharmaceutical or cosmetic composition which does not cause secondary reactions and for example allows facilitated administering of the active compound(s), increased lifetime thereof and/or efficacy in the body, increased solubility in solution or improved shelf life.

These acceptable excipients are well known and can be adapted by persons skilled in the art to the type and mode of administration of the selected active compound(s).

Advantageously, the nutritional, cosmetic or pharmaceutical composition of the invention has a superoxide dismutase content of between 0.01 and 10 weight %, for example between 0.01 and 5 weight % of superoxide dismutase(s), or between 0.01 and 1 weight % relative to the total weight of the composition.

In one embodiment, the cosmetic composition of the invention is intended for external topical use such as care products, shampoos, lotions, gels.

When the composition of the invention is intended for nutritional use, it is advantageously intended for administration via oral route. For example, the nutritional composition of the invention is in the form of a tablet, hard capsule, soft capsule, effervescent tablet, sachet or stick to be diluted, chewing gum, beverages, juices, yoghurt, confectionery, biscuit or bars.

When the composition of the invention is intended for pharmaceutical use, it is intended for administration via topical, oral, nasal or parenteral route. For example, the pharmaceutical composition of the invention is in the form of a tablet, hard capsule, soft capsule, effervescent tablet, sachet or stick to be diluted, syrup, elixir, herbal tea, chewing gum, spray, aerosol or solution for injection.

In one preferred embodiment, the pharmaceutical composition of the invention is intended for administration via oral route.

For the oral route, the composition of the invention can be efficiently protected against gastric juices by coatings well known to persons skilled in the art, in particular coatings of plant fatty substances (see patent FR 2 822 381) or in particles of modified starch (see international application WO2006/030111).

Therefore, the compositions of the invention can be efficiently delivered via oral route and induce an increase in the synthesis of endogenous superoxide dismutase and glutathione peroxidase in every organ where oxidative stress is present, allowing efficient and constant combatting of pathologies related to oxidative stress.

In one particular embodiment, the pharmaceutical, cosmetic, veterinary or nutritional composition of the invention contains another active ingredient, advantageously another antioxidant.

The present invention also concerns a composition of the invention for use thereof as medicinal product.

Administration of SOD, or of the SOD mixture of the invention induces endogenous cell synthesis of SOD and of an enzyme in the second defence line: glutathione peroxidase. This enables the cell and the body to combat oxidative stress efficiently and sustainably. The medicinal product is therefore preferably intended to treat or prevent diseases related to oxidative stress and/or inflammation and/or to reinforce the action of other antioxidants used in animals. The medicinal product can also be intended to stimulate the vitality of animal cells.

In one embodiment, the composition of the invention for use as medicinal product is administered in association with another medicinal product. Preferably, this medicinal product is also intended to treat or prevent diseases related to oxidative stress and/or inflammation and/or to reinforce the action of other pharmaceutical molecules, and in particular other antioxidants used in animals. This medicinal product may also be intended to treat orphan diseases.

In one embodiment of the invention, the animal is selected from among the group of livestock animals, pets or laboratory animals, advantageously selected from among the group formed by porcine, bovine, Equidae, ovine, caprine animals, Cervidae, poultry, rabbits, aquaculture animals, fish, shellfish, advantageously shrimps and crabs, reptiles, advantageously tortoises and crocodiles, birds, felines, rodents, advantageously mice, rats and guinea pigs and Canidae, advantageously from among ruminants and monogastric animals.

In such animals, oxidative stress-related disease and/or inflammation is preferably selected from among chronic inflammations such as osteoarthritis, tendinitis, laminitis, auto-immune diseases, mastitis leading to the presence of cells in milk, embryonic mortality in animal reproduction, stress effects caused by temperature change, diet, breeding density, habitat, during competitions, under intense and/or extended exercise e.g. when working, walking or running, vaccinations, intensive feeding or during transition periods such as giving birth, laying, weaning, tank change, initiation of rearing e.g. placing in nurseries, brooding areas, stocking with young fish, lactation, transport, batching, reproduction disorders of animals e.g. poor maturing of follicles and/or ova, specific and non-specific immunity disorders of animals, in particular microbial, viral, parasitic aggressions and/or lack of body resistance to disease, and oxidative overload. In another embodiment of the invention, the animal is man. In this case, the diseases related to oxidative stress and/or inflammation are advantageously selected from among the group formed by allergies e.g. eczema, vitiligo, lupus, disorders related to UV radiation, radiation-induced dysfunction in particular fibrosis of varying origins and cystitis, infertility, respiratory pathologies in particular asthma, anaemia, rheumatoid arthritis and osteoarthritis pathologies, in particular knee or finger osteoarthritis, genitourinary disorders in particular Peyronie's disease, problems related to ischaemia—reperfusion, pathologies of the nervous system in particular hypoxia, cardiovascular diseases in particular hypertension, obesity, Type II diabetes, cancer treatment in particular colorectal cancers or melanomas, colorectal inflammation in particular Crohn's disease, neurodegenerative pathologies in particular Alzheimer's or Parkinson's disease or amyotrophic lateral sclerosis, ulcerative colitis, sight disorders e.g. ARMD, mitochondrial dysfunction, degeneration due to an infectious agent, AIDS and hepatitis C in particular, or degeneration related to the use of a drug or exposure to a toxic chemical product e.g. pesticides. Mention can also be made of orphan diseases, cystic fibrosis, Friedreich's ataxia and alopecia. Improvement in fertility can also be cited, improved vitality of stem cells allowing better use and efficacy in cell therapies, in particular for inflammatory joint diseases, bone marrow diseases and neurodegenerative diseases such as Alzheimer's disease.

Finally, the mixture of the present invention, on account of its particularly advantageous antioxidant action, is also useful in the treatment of myopathies (for a reference linking myopathy to oxidative stress see: Functional muscle impairment in fascioscapulohumeral muscular dystrophy is correlated with oxidative stress and mitochondrial dysfunction. Turki et al, 2012. Free Radical Biology and Medicine: 1:53(5) 1068-1079).

In addition, as indicated above, the SOD mixture of the invention favourable modulates expression of the genes NPPA and RXFP1 in animals, and in man in particular.

The NPPA genes codes for a protein belonging to the family of natriuretic peptides involved in the volume of extracellular fluids and homeostasis of electrolytes. The human NPPA gene encodes a preprohormone of 151 amino acids (AA). After cleavage of the N-term signal sequence of 25AA, the resulting prohormone (pro ANP) has 126 AA and is stored in atrial secretory granules. When secreted, proANP is converted to active ANP (Atrial Natriuretic Peptide) (1).

The secretion of ANP is essentially dependent on a mechanical stimulus; it is variations in volemia which regulate ANP via variations in atrial parietal tension, thereby promoting through its action the lowering of blood pressure. ANP therefore has anti-hypertrophic action. In the event of hypertrophy or heart failure, proANP and ANP are secreted in the ventricular myocardium leading to their increase in the plasma in the event of heart disease (1).

ANP plays a role in the regulation of heart electrophysiology by acting on the autonomic nervous system, by inhibiting sympathetic activity, activating parasympathetic activity and causing specific regulation of the cardiac ion channels (2). The NPPA gene forms a therapeutic target for selective regulation of the progression of cardiovascular diseases (1), since it is stimulated in the event of heart disease (3). Diseases associated with the NPPA gene are acute myocardial infarction, diseases of the mitral valve, heart failure, hypertension, fluid overload or cardiac hypertrophy. Mutations of this gene are associated with atrial fibrillation (2). Higher NPPA expression in the heart is involved in the pathophysiology of hypertension and heart failure (3).

The RXFP1 receptor, also known as LGR7 (Leucine-rich-repeat containing G-protein-coupled Receptor 7), is a polypeptide of 757 amino acids belonging to the insulin superfamily. It is a transmembrane receptor binding to relaxin with high affinity. It is coupled to a G protein and has 7 transmembrane domains. In addition, its ectodomain has 10 “leucine-rich-repeats” (LRR) and consists of class A lipoproteins: LDLa (Low Density Lipoprotein a) as terminal amine, which impacts maturing of the receptors, their positioning on the cell surface and signalling of relaxin [L]. This receptor is therefore at the root of cell signalling and signal transduction. Relaxin receptors are found in reproductive tissue, the brain, kidneys, heart, lungs in which the action of relaxin has been evidenced (4). In the heart, the RXFP1 receptors are found firstly in the atrium rather than in the ventricle. However, it has been established that expression of the mRNAs of RXFP1 also takes place in the ventricle of rats, mice and human beings. The high density of RXFP1 in the atrium is linked to the inotropic and chronotropic response of relaxin. Further to activation of the RXFP1 receptors, and transducing of the signal, relaxin will have different effects. The function of relaxin is not confined to the reproductive system, this peptide affects the heart function and inter alia takes part in the regulation of blood pressure, blood flow, fluid balance (5). Relaxin has antifibrotic, antihypertrophic, anti-inflammatory and vasodilator effects. It also reduces arterial pressure and increases heart rate and can also stimulate regeneration of the myocardium (4) (5).

Levels of RXFP1 mRNA are reduced in the atrium and left ventricle of MI rats (myocard Infarct) (4).

Therefore, the SOD mixture of the present invention is advantageously used for the treatment of cardiovascular diseases, in particular for acute myocardial infarction, diseases of the mitral valve, heart failure, hypertension, fluid overload, heart hypertrophy, hypertension and heart failure, in particular in humans.

The SOD mixture of the invention is also advantageously used for the treatment of obesity and arteriosclerosis.

Additionally, it has been ascertained that the SOD mixture of the present invention is useful for the treatment of herpes, in particular labial herpes, in particular in humans.

The present invention also concerns a composition of the invention for use thereof as nutritional, food or cosmetic composition. In this case, the composition is intended to combat the daily effects of oxidative stress such as: fatigue, stress, anxiety, joint problems, recovery after sports, male or female fertility problems, erection, sight problems, healing, cellulitis, UV radiation, acne.

### FIGURES

FIG. 1: Determination of different SODs derived from the F1 hybrid variety of the MA 7950 melon (schematic illustration of the result obtained after detection on acrylamide gel). Total SOD activity (well 1), with 2 mM KCN (well 2), with H₂O₂ (well 3).

FIG. 2: electrophoretic profile of the different SODs derived from the F1 hybrid variety of the MA 7950 melon (schematic illustration of the result obtained after detection on IEF gel); from left to right: MA 7950 melon (wells 1 and 2), water melon (wells 3 and 4), peach (wells 5 and 6) and nectarine (wells 7 and 8).

FIG. 3: electrophoretic profile of the different SODs derived from the F1 hybrid variety of the MA 7950 melon (schematic illustration of the result obtained after detection on IEF gel); from left to right: MA 7950 melon (wells 1 and 2), Canari melon (wells 3 and 4), apricot (wells 5 and 6) and cherry (wells 7 and 8).

FIG. 4: Photograph of two melons just after picking (t0). On the left, a common F1 hybrid melon, on the right a melon derived from the F1 hybrid melon called MA 7950.

FIG. 5: Photograph of the two same melons taken 7 days later. On the left the F1 hybrid melon of common type, on the right the melon derived from the F1 hybrid variety of the melon known as MA 7950.

### EXAMPLES

The following examples are given for illustration purposes and do not in any manner limit the invention.

### Example 1. Specificity of the F1 Hybrid Variety of Melon called MA 7950

A simple experiment was conducted to demonstrate the particular properties of the melons derived from the F1 hybrid variety called MA 7950, and its antioxidant activity in particular.

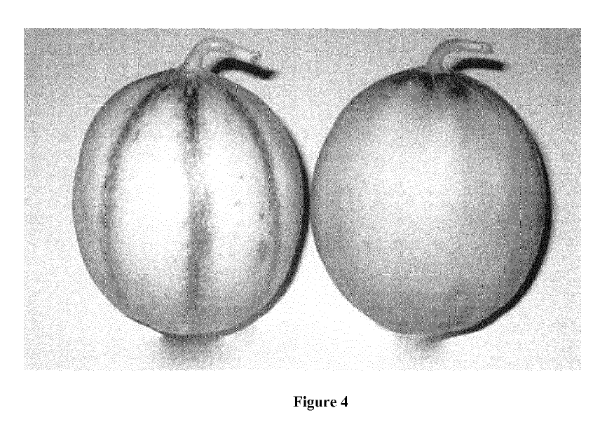

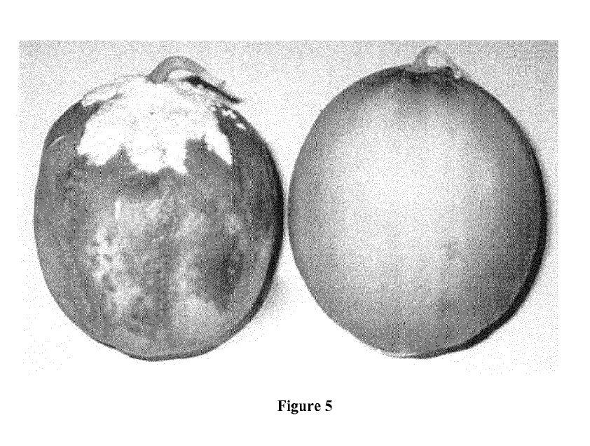

Two melons, one of common type and the other derived from the F1 hybrid variety of melon called MA 7950, were placed in a room at ambient temperature and under atmospheric pressure at D1 (FIG. 4). The melons were kept in this room under the same conditions (ambient temperature, atmospheric pressure) for 7 days (FIG. 5: photograph of the same melons taken at D8). After 7 days, it can be clearly seen that the F1 hybrid melon of common type is in an advanced state of degradation, while the melon derived from the F1 hybrid variety of the melon called MA 7950 still has a good outer appearance. The twofold higher content and composition of the SOD mixture of the F1 hybrid variety of the MA 7950 melon provide the cells of the melon with more efficient resistance against the natural degradation process.

### Example 2. Preparation of the Extract and Purification of the Different SODs, Assay of SOD Activity

5 g of pulp of Cucumis melo of the F1 hybrid variety called MA 7950 or the cells thereof were cold crushed in a mortar. A volume of 50 mM phosphate buffer (pH: 7.8; EDTA 1 mM; 5% glycerol) equivalent to 3 times the plant mass was added. After homogenisation, the suspension was centrifuged at 5000 g at 4° C. for 30 minutes. The supernatant was recovered and filtered. This crude extract was used for purification of the different SOD forms. The different SODs were purified applying a technique well known in the art: IMAC (Immobilised Metal Affinity Chromatography). For this method, copper was used as metal ion immobilised on the column; attaching of the proteins is dependent on their charge and therefore correlates with the presence of amino acids such as tryptophan, histidine, cysteine. SODs are known to have a high histidine content, justifying the use of IMAC.

The SODs derived from the extract of the MA 7950 melon were purified on FPLC apparatus (Pharmacia Amersham) on a Superose HR 10/2 column or Hitrap chelating column (Pharmacia-Biotech). 3 ml of sample were injected via a superloop into the column previously treated with a solution of CuSO4 (600 ml, 23 mmol/ml) and equilibrated with 10 ml of 0.05 M potassium phosphate buffer, pH 7.8. Elution of the SODs was conducted at a constant rate of 1 ml/min with a 10% linear gradient (reached in 10 min) of 0.75 M NH₄Cl solution. The remaining Cu-protein complexes were removed with 5 ml of aqueous 1M EDTA solution. 2 ml fractions containing the different SODs were collected, assayed and immediately desalted on an Amicon PM 10 chamber with 4 volumes of 0.05 M phosphate buffer, pH 7.8, then freeze-dried.

### Example 3. Determination of the Different SOD Forms and Molecular Weights

SOD activity was detected on acrylamide gel under native conditions (conditions under which the protein remains in the native state i.e. such as it is in the cell, as opposed to denaturing conditions in which the protein is linearized) following the method of Beauchamp and Fridovich Anal. Biochem. 44:276-82 (1971). It is based on SOD-inhibited reducing of Nitroblue Tetrazolium (N 55 14 Sigma-Aldrich, France). After the reaction, the different bands corresponding to SOD activity appear in white against a background of dark blue gel. Samples of the mixture of purified SODs (45 μl of solution A+15 μl migration buffer) were deposited in different wells of an electrophoresis gel (4%/10% acrylamide). Migration time was 75 min at 70 mA and 300V. At the gel resolving step, which migrated under non-denaturing conditions, the gel was cut into 3 portions. The activity of the first was determined following the protocol below, the second was preincubated with 2 mM KCN and the activity determined, finally the third was preincubated with 5 mM H₂O₂ and the activity determined.

### SOD Activity Identification Conditions:

The activity of the purified SODs was measured with a SOD assay kit (19160, Sigma-Aldrich, France). This total SOD activity was higher than 500 IU SOD/mg of proteins and the protein content of the purified extract was higher than 70% (Bradford method, Anal. Biochem. 72: 248-54, 1976). After migration, the gels were immersed:
for 150 min in K+ phosphate buffer (50 mM; pH 7.8) containing 2 mM NBT;
then for 15 min in K+ phosphate buffer (50 mM; pH 7.8) containing 28 mM TEMED and 0.0028 mM riboflavin (a 50 mg solution of riboflavin was prepared in 1 ml of phosphate buffer).

The gels were then rinsed in K phosphate buffer solution (50 mM; pH 7.8). The electrophoresis gels were subsequently digitized using the Perfect Image photographic capture module and analysed on a Gel Analyst module (Claravision, 2000, France).

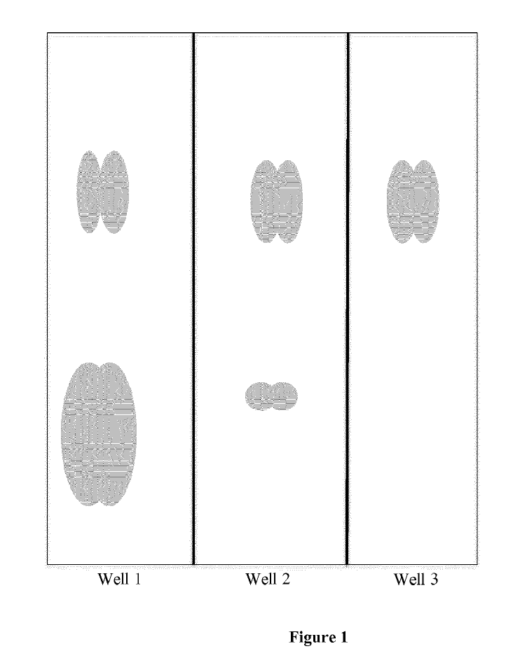

As can be seen in FIG. 1, in the absence of inhibitors, the SOD mixture exhibits two thick bands of SOD activity (well 1), one located towards the top part of the gel indicating proteins of high molecular weight (which migrate less quickly than the proteins of low molecular weight) and the other towards the bottom part of the gel indicating activity due to one or more SODs of low molecular weight.

In the presence of 2 mM KCN (well 2), only Cu/Zn-SOD is inhibited, this corresponding to the band located at the bottom part of the gel and representing the largest portion of this activity band.

In the presence of 5 mM H₂O₂ (well 3), Fe-SOD and Cu/Zn-SOD are inhibited. The only remaining band on the gel (top part) corresponds to Mn-SOD (insensitive to KCN and H₂O₂). Mn-SOD represents part of the SOD activity band located at the top of the gel, the other part is Fe-SOD inhibited by H₂O₂. The other form of Fe-SOD is located at the bottom part of the gel close to Cu/Zn-SOD.

The intensities of the different bands of SOD activity were integrated using the Gel Analyst module (Claravision, 2000, France), and the ratio of each SOD isoform thus calculated:

| Intensity of | SOD without | | |
|---|---|---|---|
| each band | inhibitors | +KCN | +H₂O₂ |
| 1 | 177 160 | 173 721 | 62 124 |
| 2 | 442 524 | 43 594 | / |
| Total | 619 684 | 217 315 | 62 124 |
| Results | SOD without | | |
| as % | inhibitors | +KCN | +H₂O₂ |
| 1 | 28.6 | 28, | 10 |
| 2 | 71.4 | 7 | / |
| Total | 100 | 35 | 10 |

For Mn-SOD (band 3), the intensity of the band is 62 124 for a total intensity of 619 684 (SOD without inhibitors), Mn-SOD therefore represents 10% of total SOD activity. For Cu/Zn-SOD, the intensity of the band is (442 524-43 594)/619 684 and corresponds to 65% of total activity. Finally, Fe-SOD corresponds to 25%, one isoform represents 7% (close to Mn-SOD) and the other 18% (close to Cu/Zn-SOD).

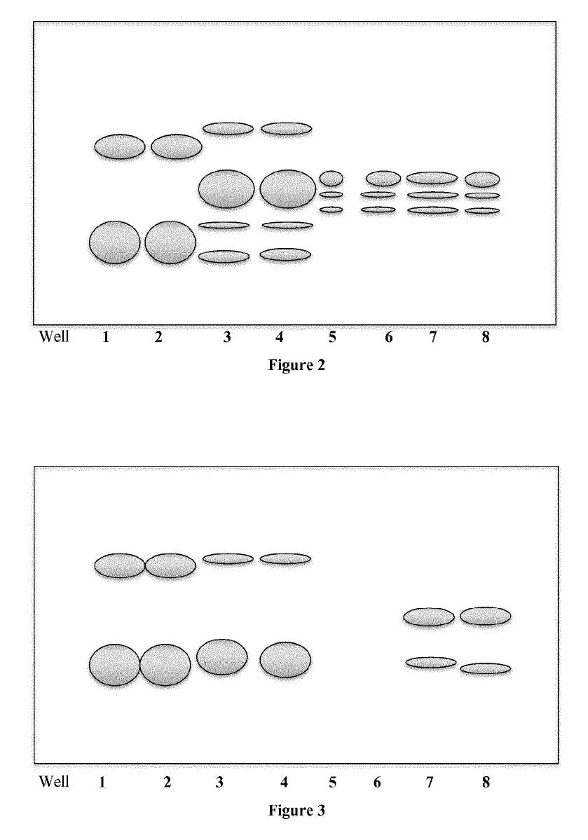

The above analyses performed on a SOD mixture extracted from the F1 hybrid variety of the Cucumis Melo MA 7950 melon, were carried out under the same conditions on other SODs derived from different fruit: Anasta melon, Spanish melon (Canari), Clipper melon (descendant of the F1 hybrid variety of Cucumis Melo 95LS444), water melon, peach, nectarine, cherry (FIGS. 2 and 3).

As can be seen in FIGS. 2 and 3, the electrophoresis profiles of the SOD activities derived from different types of fruit differ largely from those of a mixture of purified SODs extracted from the F1 hybrid variety of the Cucumis Melo melon MA 7950 (the furthest to the left).

The percentages of each form after use of inhibitors (2 mM KCN and 5 mM H202) are given in Table 1.

TABLE 1
Proportion of the different SOD forms.

| | Cu/Zn-SOD | Mn-SOD | Fe-SOD |
|---|---|---|---|
| Melon MA 7950 | 65% | 10% | 25% |
| Water melon | 66% | 18% | 16% |
| Canari melon | 83% | 9% | 8% |
| Anasta melon | 84% | 13.5% | 2.5% |
| Clipper melon | 25% | 60% | 15% |
| Nectarine | 81.5% | 0% | 18.5% |
| Peach | 88% | 0% | 12% |
| Cherry | 100% | 0% | 0% |
| Apricot | 0% | 0% | 0% |

On these same electrophoresis gels, molecular weight markers (proteins of known molecular weights) were placed in the first well of the gel using a marker kit (M 3913, Sigma-Aldrich, France) to calibrate the gel for molecular weight and to determine the molecular weight of the different SODs by comparison.

The same analyses were performed on mixtures of purified SODs from other fruit, only on fruit containing 3 forms of SOD: water melon, Spanish melon (Canari), other melon variety (Anasta).

The molecular weights of the different SODs are given in Table 2.

TABLE 2
Molecular weights of different SOD forms extracted from the F1
hybrid variety of Cucumis Melo MA 7950, from a water melon,
Canari melon, Anasta melon and Clipper
melon (descendant of the F1 hybrid variety
of the Cucumis Melo 95LS444 melon).

| | Cu/Zn SOD | Mn SOD | Fe SOD |
|---|---|---|---|
| Melon MA 7950 | 31 800 | 80 600 | 32 200 |
| | | | 79 800 |
| Water melon | 41 600 | 72 200 | 56 400 |
| Canari melon | 44 700 | 65 900 | 43 700 |
| Anasta melon | 42 600 | 62 700 | 44 700 |
| Clipper melon | 40 000 | 95 000 | 30 000 |

It can easily be seen that the mixture of purified SODs extracted from the F1 hybrid variety of Cucumis Melo MB17415 is unique (compared with the other SODs derived from different fruit), in particular on account of the presence of the second isoform of Fe-SOD. It contains 3 classes of SOD (differentiated through their metal group) and 4 isoforms:
Cu/Zn-SOD represents 65% of total SOD activity and has a molecular weight of 31 800 Da.
Mn-SOD represents 10% of total SOD activity and has a molecular weight of 80 600 Da.
2 different forms of Fe-SOD represent 25% of total activity: One Fe-SOD of 32 200 Da represents 18% of total SOD activity. The other Fe-SOD of 79 800 Da represents 7% of total SOD activity.

### Determination of the Isoelectric Points of the Different SODs

This analysis technique is of interest to obtain additional information on the pHi of the isoforms of the SODs purified from melon. It is therefore an activity technique. The separation of the SODs as a function of their charge is obtained by isoelectric focusing (IEF). The SODs migrate in an electric field and become immobilised when the pH environment causes them to lose their net charge. The isoelectric points of the different SOD forms can be determined on IEF gels by comparison with markers of known isoelectric point (IEF markers 3.6-9.3, Reference 56733, Sigma-Aldrich, France)

The samples deposited on gels (30% acrylamide/acrylamide bis, 50% glycerol, ampholines pH 3.5-10 Amersham, ammonium persulfate and Temed) migrate at 300 V and 20 mA in a sample buffer (glycerol: 75% v/v, ampholines: 2% v/v) and a migration buffer (25 mM NaOH for the cathode and 25 mM CH3COOH for the anode). Isoelectric point markers are also deposited on a gel well. Detection of the bands of SOD activity is obtained as described in paragraph 2. By comparison with the bands of the pHi markers, the pHi values of the different isoforms are determined. For the Anasta melon, the low proportion of Fe-SOD (2.5%) does not allow identification of the different pHi values. The isoelectric points of the different SOD forms of the 3 varieties (MA 7950; water melon, Canari melon) are given in Table 3.

TABLE 3
Isoelectric points (pHi) of the different SOD forms
extracted from the F1 hybrid variety of Cucumis Melo
MA 7950, water melon and Canari melon.

| | Cu/Zn SOD | Mn SOD | Fe SOD |
|---|---|---|---|
| Melon MA 7950 | 4.3 | 4.1 | 4.4 |
| | | 4.4 | 4.7 |
| | | 5.3 | |
| | | 5.5 | |
| | | 5.7 | |
| | | 5.85 | |
| | | 6.1 | |
| Water melon | 4.6 | 4.25 | 4 |
| | | 4.7 | 4.7 |
| | | 5.2 | |
| | | 5.8 | |
| | | 6 | |
| | | 6.1 | |
| Canari melon | 4.4 | 4.4 | 4 |
| | | 5.7 | |
| | | 5.8 | |
| | | 5.9 | |
| | | 6 | |

It is noted that the mixture of purified SODs, extracted from the F1 hybrid variety of Cucumis Melo MA 7950 and characterized by their metal group, molecular weight and pHi, is unique; it contains:
Cu/Zn-SOD represents 65% of total SOD activity, has a molecular weight of 31 800 Da and pHi of 4.3;
Mn-SOD represents 10% of total SOD activity, has a molecular weight of 80 600 Da and has 7 isoforms of different pHi values between 4 and 6;
2 different forms of Fe-SOD represent 25% of total activity: One Fe-SOD of 32 200 Da represents 18% of total SOD activity. The other Fe-SOD of 79 800 Da represents 7% of total SOD activity. They have a pHi of 4.4 and 4.7.

In 1 mg of the mixture of the invention, there are about 0.71 mg of Cu/Zn SOD, 0.06 mg of Mn-SOD and 0.23 mg of Fe-SOD.

### Example 4. Efficacy of the Mixture of Purified SODS of the Invention on a Mmarker of Oxidative Stress: the Superoxide Anion (O₂°—) Produced by NADPH Oxidase, and on an Inflammation Marker: TNF α

In macrophages, the use of molecules such as interferon gamma or LPS induce an oxidative burst (over-production of the superoxide radical due to stimulation of NADPH-oxidase) and an inflammatory response (increased production of TNF α and of interleukins).

The effect was evaluated of the different doses of a mixture of purified SODs of the invention on the production of the superoxide radical (O₂.—) and on the production of an inflammatory cytokine (TNF α), on a cell line of murine RAW 264.7 macrophages.

The cells were cultured in an RPMI medium (Roswell Park Memorial Institute Medium) to which were added: fcetal calf serum (10%, Life Technologies), a penicillin-streptomycin antibiotic solution (1%, Life Technologies) and fungizone (0.25%, Life Technologies) in T 175 cm² culture dishes (Dutscher, Brumath, France) in a humid atmosphere enriched with 5% CO₂ at 37° C. The cells were counted and passaged every three days using a Thoma coverslip and then distributed in culture dishes at a concentration of 250 000 cells/ml. The cells were subsequently incubated with a mixture of purified SODs of the invention (0 to 100 IU SOD/ml ) for 12 hours. Thereafter, they were detached from the support and suspended at a concentration of 10⁶ cells/ml in RPMI. 890 μl of this suspension were placed in the presence of 100 μl of 1 mM lucigenin and incubated at 37° C. for 30 minutes. After these 30 minutes, and at the time of measurement, 10 μl of 10⁻⁷ M PMA were added (Phorbol-12-Myristyl-13-Acetate, Sigma Chemical; St louis Mo.; USA) to measure the production of the superoxide radical. To stimulate the production of TNF-α, lipopolysaccharide (LPS) was added to the wells 12 h before this measurement.

The production of O₂°—was measured using a chemiluminescence technique (Chen et al., Am. J. Renal. Physiol. 289 F 749-53 2005) in the presence of lucigenin (10 μM), a bioluminescence probe specific to the superoxide anion. In brief, in the presence of O₂°—, lucigenin changes over from a fundamental state to an excited state. On its return to the fundamental state, lucigenin emits photons. The number of photons emitted is proportional to the formation of O₂°—. The intensity of luminescence is recorded by a luminescence microplate reader (Synergy 2 Biotek, USA). The results are expressed in hits/mg of proteins.

The production of TNF α in the macrophages is measured by ELISA with a DY 510 analysis kit (R&D system, Mineapolis, USA). The results are a mean of 3 independent repeats.

As can be seen in Tables 4 and 5, the mixture of SODs derived from the MA 7950 line modulate the oxidative and inflammatory response of the line of murine macrophages by inhibiting the production of the superoxide anion by NADPH oxidase, and the production of TNFα.

TABLE 4
Effect of different doses of a mixture of purified SODs
extracted from the F1 hybrid variety of Cucumis Melo
MA 7950 on the production of the superoxide anion.

| (SOD (UI/ml) | 0 | 5 | 10 | 50 | 100 |
|---|---|---|---|---|---|
| % activation | 100 ± 5 | 79.5 ± 5.5 | 66.4 ± 3.2 | 30.5 ± 2.5 | 19.6 ± 2.4 |

TABLE 5
Effect of different doses of the mixture of MA 7950
SODs on the production of TNF α

| (SOD (UI/ml) | 0 | 5 | 10 | 50 | 100 |
|---|---|---|---|---|---|
| TNF α (pg/ml) | 721 ± 12 | 454 ± 17 | 166 ± 16 | 69 ± 7 | 59 ± 5 |

### Example 5: Efficacy of the Mixture of Purified SODs of the Invention on the Endogenous Synthesis of Antioxidant Enzymes

A mixture of purified SODs of the invention was encapsulated following the method described in international application WO2006030111, to allow use via oral route. 10 male Wistar rats (bred by Janvier, Le Genest-St-Isle, France) aged 3 weeks were used.

On arrival, they were randomly divided into groups and housed in plastic cages in a controlled environment at a temperature of 23±1° C., hygrometry of 70% and 12-hour photoperiod (12 h light/12 h darkness). The rats were handled in accordance with NIH guidelines (National Research Council).

The animals had free access to food and water. Food consumption was measured daily and the body weight of the animals was recorded every other day.

The animals were separated into two groups of 5.

One group received a standard diet (EF R/M control E 15000-00, SSNIFF, Germany)

The other group was given the standard diet (EF R/M control E 15000-00, SSNIFF, Germany) as well as a dose of 4 IU SOD (or U/mg SOD) of the mixture of the invention per day for 28 days.

At the end of the experimental period of 28 days, the animals were anesthetized with an intraperitoneal injection of pentobarbital. The liver was infused with 0.15M NaCl, then sampled and stored at −80° C.

The expression of the SOD and GPx antioxidant enzymes was determined by Western-blot.

The results are given in Table 4. As can be seen, the ingestion of the mixture of SODs derived from the MA 7950 melon induces the expression of the antioxidant enzymes SOD and GPx in Wistar rats in the absence of oxidative stress.

TABLE 6
Quantification of the expression of SOD and glutathione
peroxidase (GPX) in the liver of male Wistar rats

| | SOD | GPX |
|---|---|---|
| control animals | 100 | 100 |
| animals treated with a mixture of | 235 ± 8 | 161 ± 5 |
| purified SODs of the invention | | |

In a situation of oxidative stress, this stock of antioxidant proteins can be activated and used, allowing a better cell response to oxidative stress.

### Example 6: Effect of the SOD Mixture of the Invention on the Expression of the NPPA and RXFP1 Genes

The effect of the mixture of purified SODs of the invention on the genes involved in cardiac hypertrophy was evaluated on a model of spontaneously hypertensive rats (SHR). These spontaneously hypertensive rats have cardiac hypertrophy originating from genetic cross breeding, and can be used on reception therefore having the advantage of being “ready-for-use”.

The different mixtures of purified SODs of the invention were encapsulated following the method described in international application WO2006/030111 to allow use via oral route.

40 male SHR rats (bred by Janvier, Le Genest-St-Isle, France) aged 3 weeks were used. On arrival, they were randomly divided into groups and housed in plastic cages in a controlled environment at a temperature of 23±1° C., hygrometry of 70% and photoperiod of 12 hours (12 h light/12 h darkness). The rats were handled in accordance with NIH guidelines (National Research Council).

The animals had free access to food and water. Food consumption was measured daily and the body weight of the animals was recorded every other day.

The animals were separated into 4 groups of 10 (groups 1, 2, 3 and 4).

Group 1 received a standard diet (EF R/M control E 15000-00, SSNIFF, Germany)

Group 2 received the standard diet (EF R/M control E 15000-00, SSNIFF, Germany) for 4 days as well as a dose of 4 IU SOD (or U/mg SOD) per day of the SOD mixture derived from the MA7950 melon of the invention.

Groups 3 and 4 received the standard diet (EF R/M control E 15000-00, SSNIFF, Germany) for 4 days and a dose of 4 IU SOD (or U/mg SOD) per day of the SOD mixture derived from the Canari melon (group 3) or Anasta melon (group 4)

After the 4 experimental days, the animals were anesthetized with an intraperitoneal injection of pentobarbital. The heart was taken, and assay of ANP and RXFP1 performed by Western blot on the left ventricle (LV).

Protein extraction from the LV was conducted on ice with 20 mM Tris buffer (pH 6.8) containing 150 mM NaCl, 1mM 1% EDTA, 20% Triton, 0.1% SDS, and 1% cocktail of protease inhibitors. After centrifuging (1500 rpm, 15 min at 4° C.), the supernatant was collected and the tissue proteins separated by SDS PAGE. An equivalent amount of proteins was deposited on 10 or 15% acrylamide gels with a concentration gel of 4% acrylamide. Migration took place in Tris-glycine-SDS buffer from Sigma-Aldrich (Saint Quentin Fallavier, France). After separation, the proteins were transferred onto nitrocellulose membranes (Whatman, Dassel, Germany).

Quantification was conducted after standardisation in the membranes by expressing the density of each band of interest relative to GAPDH in one same line. The results are expressed as changes relative to the placebo group. ** p<0.01 compared with the placebo group.

The results are given in Table 7 below.

TABLE 7
Quantification of ANP and RXFP1
expression in the heart of SHR rats.

| | ANP | RXFP1 |
|---|---|---|
| Control animals | 100 | 100 |
| Animals treated with mixture of SODs | 54 | 147 |
| derived from MA7950 melon | ** | ** |
| Animals treated with mixture of SODs | 98 | 103 |
| derived from Canari melon | (NS) | (NS) |
| Animals treated with mixture of SODs | 97 | 96 |
| derived from Anasta melon | (NS) | (NS) |

(NS): non-significant;
**: significant

As can be seen in Table 7 above, solely the ingestion of the mixture of SODs derived from the MA 7950 melon (SOD mixture of the invention) leads to modulation of the expression of the ANP and RXFP1 proteins, of which the NPPA and RXFP1 genes are involved in cardiovascular pathologies, in particular cardiac hypertrophy and obesity. This modulation, as explained above, is beneficial in the event of cardiovascular disease (reduction of ANP concentration and increase in RXFP1 concentration).

1. A mixture of superoxide dismutases of plant origin, characterized in that it is essentially consisting of 3 superoxide dismutases: a manganese superoxide dismutase, a copper and zinc superoxide dismutase and an iron superoxide dismutase in at least two isoforms, the first isoform of iron superoxide dismutase having a molecular weight of between 28 000 and 36 000 Da, the second isoform of iron superoxide dismutase having a molecular weight of between 75 000 and 85 000 Da, said mixture able to be obtained from an extract of the F1 hybrid variety of Cucumis Melo MA 7950 or the cells thereof cultured in vitro or via transfer and expression of the genes of these SODs in prokaryote or eukaryote cells.

2. The mixture of superoxide dismutases according to claim 1, characterized in that said mixture has a total SOD activity equal to or higher than 130 U/mg of said mixture.

3. The mixture of superoxide dismutases according to claim 1 or 2, characterized in that the first isoform of iron superoxide dismutase has a molecular weight of about 32 200 Da.

4. The mixture of superoxide dismutases according to any one of claims 1 to 3, characterized in that the second isoform of iron superoxide dismutase has a molecular weight of about 79 800 Da.

5. The mixture of superoxide dismutases according to any one of claims 1 to 4, characterized in that the accumulated SOD activity of the two isoforms of iron superoxide dismutase is between 20% and 26%, advantageously between 22% and 26% of the total SOD activity of the mixture.

6. The mixture of superoxide dismutases according to any one of claims 1 to 5, characterized in that the accumulated SOD activity of the two isoforms of iron superoxide dismutase is between 20% and 26% of the total SOD activity of the mixture, the activity of the copper and zinc superoxide dismutase is between 60% and 70% of total SOD activity of the mixture, and the activity of the manganese superoxide dismutase is between 7 and 12% of total SOD activity of the mixture.

7. The mixture of superoxide dismutases according to any one of claims 1 to 6, characterized in that the manganese superoxide dismutase has a molecular weight of between 70 000 and 90 000 Da, and the copper and zinc superoxide dismutase has a molecular weight of between 27 000 and 33 000 Da.

8. The mixture of superoxide dismutases according to any one of claims 1 to 7, characterized in that it is able to be obtained by grinding or pressing in an aqueous medium, preferably at a pH of 5 to 9, the F1 hybrid variety of Cucumis Melo MA 7950 or the cells thereof cultured in vitro or via transfer and expression of the genes of these SODs in prokaryote or eukaryote cells, followed by recovery of the supernatant and purification by chromatography, IMAC chromatography in particular.

9. A nutritional, cosmetic or pharmaceutical composition containing as active ingredient a mixture of superoxide dismutases according to any one of claims 1 to 7 and at least one pharmaceutically or cosmetically acceptable food-grade excipient.

10. The cosmetic composition according to claim 9, characterized in that it is intended for external topical use such as care products, shampoos, lotions, gels.

11. The pharmaceutical composition according to claim 9, characterized in that it is intended for administration via topical, oral, nasal or parenteral route, and for example is in the form of a tablet, hard capsule, soft capsule, effervescent tablet, sachet or stick to be diluted, syrup, elixir, herbal tea, chewing gum, spray, aerosol or solution for injection.

12. The pharmaceutical composition according to claim 9 or 11, characterized in that it contains another active ingredient, advantageously another antioxidant.

13. The pharmaceutical composition according to any one of claim 9, 11 or 12 for use thereof as medicinal product.

14. Pharmaceutical composition for use according to claim 13, characterized in that the medicinal product is intended to treat or prevent diseases related to oxidative stress and/or inflammation and/or to reinforce the action of other pharmaceutical molecules, and in particular other employed antioxidants, or to treat orphan diseases in animals.

15. Pharmaceutical composition for use according to claim 14, characterized in that the medicinal product is intended to stimulate cell vitality in animals.

16. Pharmaceutical composition for use according to claim 14 or 15, characterized in that the animal is human being.

17. Pharmaceutical composition for use according to claim 13, characterized in that the medicinal product is intended for the treatment of cardiovascular diseases, obesity, arteriosclerosis and labial herpes, in particular in humans.

18. Nutritional composition according to claim 9, characterized in that it is intended for administration via oral route and for example is in the form of a tablet, hard capsule, soft capsule, effervescent tablet, sachet or stick to be diluted, chewing gum, beverages, juices, yoghurt, confectionery, biscuit or bars.
